(19) United States
(12) Patent Application Publication
(10) Pub. No.: US 2025/0104851 A1
(43) Pub. Date: Mar. 27, 2025

(54) HEALTHCARE IMPROVEMENT BASED ON COMPOSITE HEALTHCARE SCORES

(71) Applicant: Hill-Rom Services, Inc., Batesville, IN (US)

(72) Inventors: Daniel C. Shirley, Raleigh, NC (US); Teresa Brasac, Miami Shores, FL (US); Anandi Mahadevan, Murrysville, PA (US)

(21) Appl. No.: 18/816,106

(22) Filed: Aug. 27, 2024

Related U.S. Application Data
(60) Provisional application No. 63/585,342, filed on Sep. 26, 2023.

(51) Int. Cl. G16H 40/20

(52) U.S. Cl. CPC G16H 40/20

(57) ABSTRACT

The present disclosure relates to a system for improving healthcare. The system includes at least one monitoring device connected to a network. The system computes at least one composite healthcare score based on the data received from the at least one monitoring device. The system identifies at least one event when the at least one composite healthcare score is below a threshold value. Furthermore, the system generates at least one recommendation for improving the at least one composite healthcare score based on the at least one event, and communicates the recommendation allowing a caregiver to view the at least one composite healthcare score and to implement the recommendation.

### BACKGROUND

Information relating to quality, safety, and efficiency associated with healthcare is often produced from patient surveys. However, the quantity of patients that voluntarily submit survey information is generally small relative to the overall patient population. Also, responses to survey questions are often subjective and biased. Given the foregoing, patient surveys do not provide an accurate understanding of the quality, safety, and efficiency of healthcare.

### SUMMARY

In general terms, the present disclosure relates to improving healthcare. In one possible configuration, a healthcare improvement system generates a recommendation to improve healthcare services based on one or more composite healthcare scores. Various aspects are described in this disclosure, which include, but are not limited to, the following aspects.

One aspect relates to a system for improving healthcare. The system includes at least one monitoring device connected to a network, at least one processing device, and a memory device storing instructions. The instructions that are executed by the at least one processing device cause the processing device to receive data from the at least one monitoring device wherein the data includes a plurality of actions performed by one or more caregivers; calculate at least one composite healthcare score by analyzing the plurality of actions performed by the one or more caregivers; identify at least one of the plurality of actions that negatively impacted the at least one composite healthcare score; generate at least one recommendation for improving the at least one composite healthcare score based on the at least one of the plurality of actions that negatively impacted the at least one composite healthcare score; and present a control on a graphical user interface that allows a caregiver of the one or more caregivers to view the at least one composite healthcare score and implement the at least one recommendation.

Another aspect relates to a method for improving healthcare. The method includes receiving data from at least one monitoring device, wherein the data includes a plurality of actions performed by one or more caregivers; calculating at least one composite healthcare score by analyzing the plurality of actions performed by the one or more caregivers; identifying at least one of the plurality of actions that negatively impacted the at least one composite healthcare score; generating at least one recommendation for improving the at least one composite healthcare score based on the at least one of the plurality of actions that negatively impacted the at least one composite healthcare score; and presenting a control on a graphical user interface that allows a caregiver of the one or more caregivers to view the at least one composite healthcare score and to implement the at least one recommendation.

Another aspect relates to a non-transitory computer readable storage medium storing instructions, which when executed by a computing device, causes the computing device to: receive an alert containing a notification that at least one composite healthcare score is below a threshold value; receive at least one recommendation for improving the at least one composite healthcare score based on an evaluation of monitoring device data; provide the at least one recommendation to a caregiver; update the at least one composite healthcare score when the caregiver complies with the at least one recommendation; and provide a second alert to the caregiver when the at least one composite healthcare score increases above the threshold value.

### DESCRIPTION OF THE FIGURES

The following drawing figures, which form a part of this application, are illustrative of the described technology and are not meant to limit the scope of the disclosure in any manner.

FIG. 1 schematically illustrates an example of a clinical care environment that includes a healthcare improvement system configured to calculate composite healthcare scores relating to healthcare provided to a patient within the clinical care environment.

FIG. 2 schematically illustrates an example of the healthcare improvement system in the clinical care environment of FIG. 1.

FIG. 3 schematically illustrates an example of a method of improving a composite healthcare score that can be performed by the healthcare improvement system of FIG. 2.

FIG. 4 schematically illustrates examples of input data that can be received by the healthcare improvement system of FIG. 2.

FIG. 5 schematically illustrates examples of system outputs that are generated by the healthcare improvement system of FIG. 2.

FIG. 6 illustrates examples of sub-scores and factors that used by the healthcare improvement system of FIG. 2 to calculate sub-composite healthcare scores.

FIG. 7 illustrates an example of a communications device displaying a composite healthcare score and a plurality of sub-composite healthcare scores that can be generated by the healthcare improvement system of FIG. 2.

FIG. 8 illustrates an example of a communications device displaying recommendations for improving the composite healthcare scores illustrated in FIG. 7.

FIG. 9 illustrates an example of a communications device displaying comparisons of efficiency scores between a caregiver and a benchmark caregiver in a similar environment.

FIG. 10 illustrates an example of composite healthcare scores being calculated for different hierarchical levels of a clinical care environment and compared to hierarchical levels of a benchmark clinical care environment.

FIG. 11 illustrates an example of a method of reporting one or more composite scores that can be performed by the healthcare improvement system of FIG. 2.

FIG. 12 illustrates a schematic view of multiple clinical care environments collecting data from a plurality of medical devices for storage in a database.

FIG. 13 illustrates a schematic view of accessing the data collected and stored in FIG. 12 for viewing one or more composite healthcare scores associated with the clinical care environments.

### DETAILED DESCRIPTION

Composite healthcare scores are a summary measurement that combines a plurality of individual metrics related to healthcare quality and performance into a single value. This single value provides a holistic view of the quality and effectiveness of healthcare services rendered at a clinical care environment. Composite healthcare scores can be used for evaluating the performance of healthcare providers, facilities, and systems, while fostering transparency, enabling informed decisions from patients, and allowing healthcare facilities to undergo improvement initiatives. Furthermore, composite healthcare scores can impact a clinical care environment's reimbursement for services rendered and employee retention.

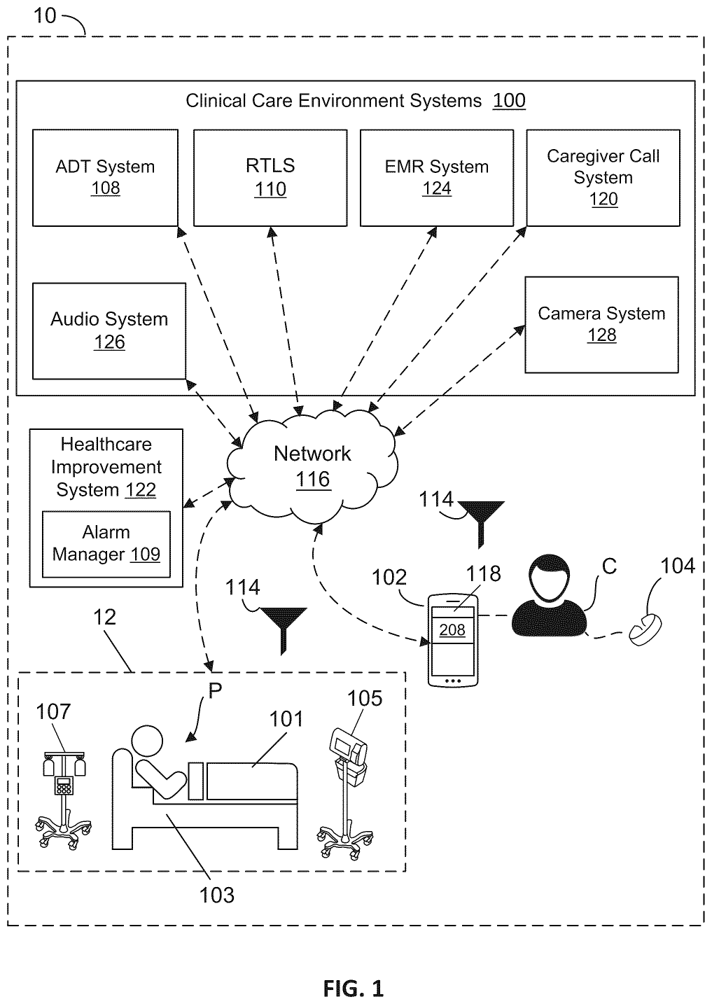

FIG. 1 schematically illustrates a healthcare improvement system 122 that improves quality, safety, and efficiency within a clinical care environment 10. As will be described in more detail below, the healthcare improvement system 122 collects data from one or more clinical care environment systems 100 and calculates one or more composite healthcare scores based on the collected data. The one or more composite healthcare scores relate to healthcare services provided within the clinical care environment 10.

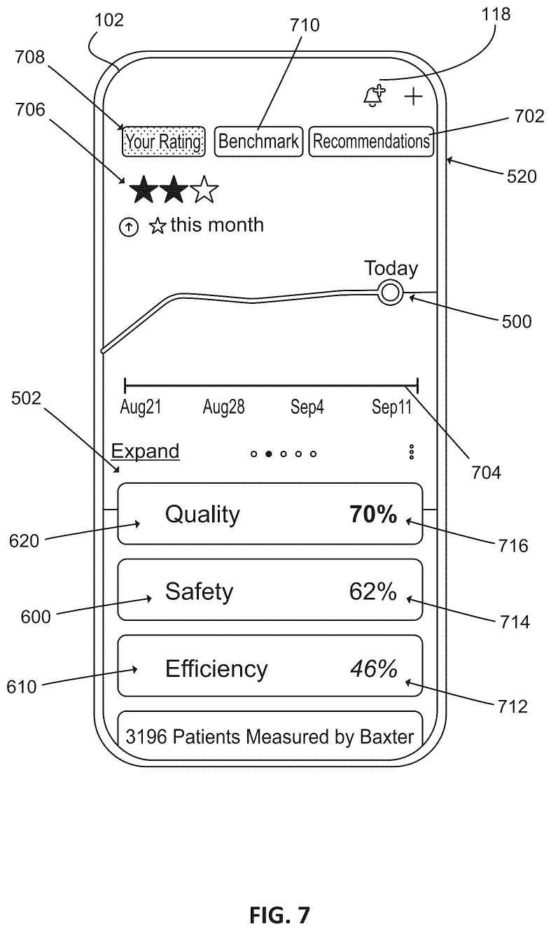

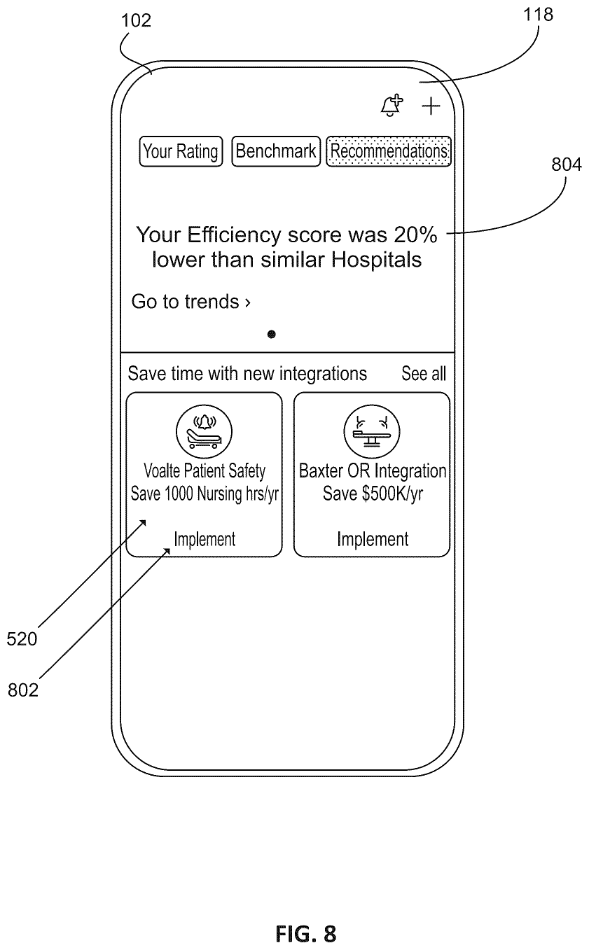

In some examples, the healthcare improvement system 122 calculates composite healthcare scores, compares the composite healthcare scores to a threshold value, and identifies any composite healthcare scores that are below the threshold value. In further examples, the healthcare improvement system 122 determines events that negatively impacted one or more of the composite healthcare scores. In certain examples, the healthcare improvement system 122 determines events that negatively impacted composite healthcare scores that are less than a threshold value. Further yet, the healthcare improvement system 122 generates an alert and/or recommendation for improving the composite healthcare scores. The alert and/or recommendation can include presenting a control on a graphical user interface that allows a caregiver C to view the composite healthcare scores and implement the recommendation (which is illustrated and described in further detail with respect to FIGS. 7-8). The healthcare improvement system 122 receives data from clinical care environment systems 100 such as an admission, discharge, transfer (ADT) system 108, a real-time locating system (RTLS) 110, an electronical medical record (EMR) system 124, a caregiver call system 120, an audio system 126, and a camera system 128, each of which will be described in great detail below.

In certain examples, the clinical care environment 10 is a clinical care environment such as a hospital, a nursing home, a rehabilitation center, a long-term care facility, and the like. Individuals located within the clinical care environment 10 can include patients P and caregivers C. As shown in FIG. 1, the patient P is located in a patient environment 12, which can include a room or other designated area within the clinical care environment 10. For example, the patient environment 12 can include a patient room, a department (e.g., emergency department), clinic, ward, or other area within the clinical care environment 10. In certain examples, the patient environment may include one or more monitoring devices for monitoring the patient P, including a patient bed 101, a spot monitor 105, or an infusion pump 107.

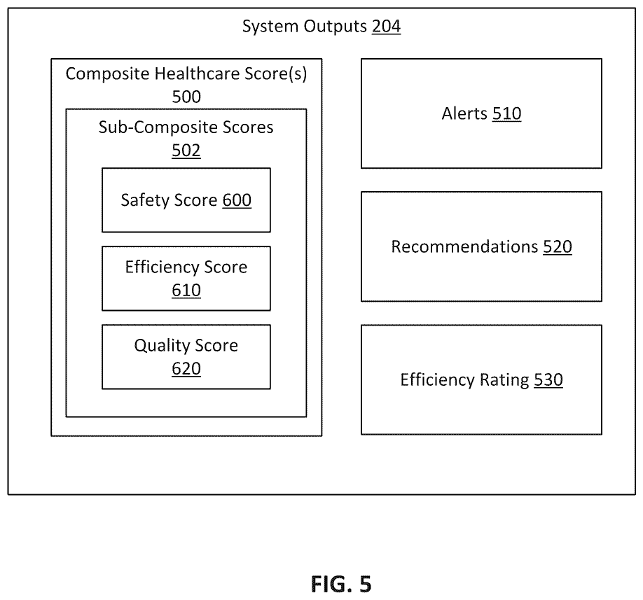

As discussed below with reference to FIGS. 5 and 8, the one or more monitoring devices within the patient environment 12 may issue alarms when the patient P requires attention from the caregiver C. Further, it is desirable to limit the number of false alarms (also known as nuisance alarms and/or nuisance alerts) that are issued from the one or more monitoring devices. The healthcare improvement system 122 includes an alarm manager 109 for managing alarms that are triggered and communicated to caregivers from the one or more monitoring devices within a patient environment 12. In one example, the one or more monitoring devices within the patient environment 12 may be too sensitive, meaning an event detection threshold is set too low such that too many false alarms are issued. The alarm manager 109 can automatically adjust the event detection threshold for a monitoring device to reduce a quantity of events detected by the monitoring device to reduce false alarms and mitigate alarm fatigue when the monitoring device issues a number of alarms that exceed a threshold. In some examples, the alarm manager 109 uses artificial intelligence such as machine learning to classify alarms as either true alarms or false alarms based on the data received from the monitoring devices in the patient environment 12. Additionally, in some further examples, the alarm manager 109 uses artificial intelligence to automatically adjust one or more event detection thresholds for one or more monitoring devices in the patient environment 12 to reduce false alarms triggered by the one or more monitoring devices, and thereby mitigate alarm fatigue in the patient environment 12.

One or more caregivers C operate a communications device 102 on which the caregiver may receive alerts and/or recommendations from the healthcare improvement system 122. Examples of the communications devices 102 can include smartphones, tablet computers, or other type of portable computing devices. In further examples, the communications devices 102 can include workstation computers. In some examples, a composite healthcare score calculation program 208 can be downloaded on the communications devices 102 to facilitate healthcare improvement. In further examples, the composite healthcare score calculation program 208 can be a web-based or cloud-based application that is accessible on the communications devices 102.

In certain examples, the healthcare improvement system 122 can provide communication channels allowing the caregivers C, administrators, or patients P to leave video, audio, or text messages to communicate during an emergency or discuss a patient's condition. As discussed below with reference to FIGS. 5-11, the composite healthcare scores can influence alerts, recommendations, and any follow-up actions required to monitor caregivers C or patients P. In some examples, the healthcare improvement system 122 is an extension of the Voalte® platform available from Hillrom®.

Data that is collected from one or more clinical care environment systems 100 (described in further detail below) within the clinical care environment 10 can be transmitted to the healthcare improvement system 122 across a communications network 116. As shown in FIG. 1, the communications devices 102 used by the caregivers C are all connected to the communications network 116. The communications network 116 can include any type of wired or wireless connections or any combinations thereof. Examples of wireless connections include Wi-Fi, Bluetooth, and broadband cellular networks including 4G or 5G. In some examples, the communications network 116 includes a broadband cellular network. In some examples the communications network 116 includes the Internet.

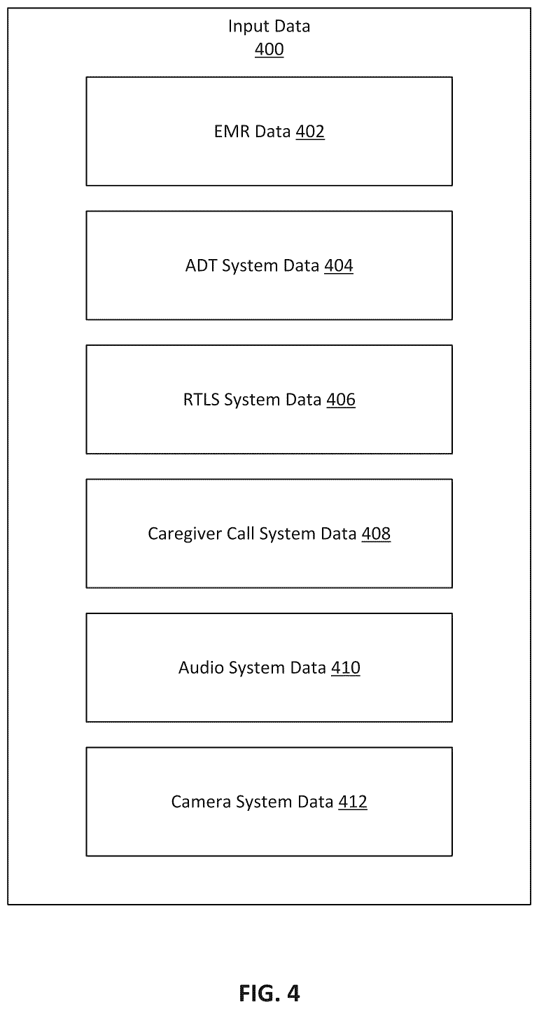

The healthcare improvement system 122 receives input data 400 (as shown in FIG. 4) over the communications network 116 that is generated by the one or more clinical care environment systems 100. The healthcare improvement system 122 analyzes the input data 400 and generates system outputs 204 (as shown in FIGS. 5-10) that are transmitted to one or more communications devices 102 via the communications network 116. As shown in FIG. 5, the system outputs 204 may include one or more sub-composite healthcare scores 502 that are dependent upon a variety of sub-scores and factors, alerts 510, and recommendations 520. Examples of the sub-composite healthcare scores 502, the alerts 510, and the recommendations 520 are described and illustrated in further detail with respect to FIG. 5.

In certain examples, a caregiver C can manually monitor composite healthcare scores that pertain to the caregiver C and request an alert and/or one or more recommendations from the healthcare improvement system 122 via the one or more communications devices 102.

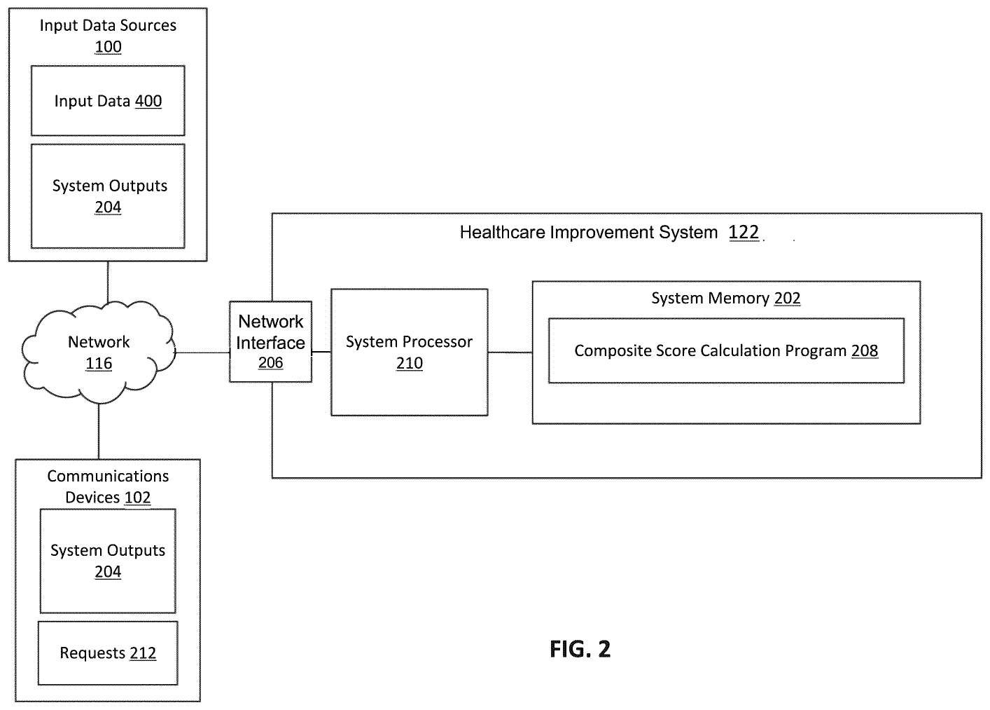

In certain examples, and as described in further detail with respect to FIGS. 5-9, the alert is a notification provided to a caregiver C from the healthcare improvement system 122 via the one or more communications devices 102 to provide additional information pertaining to one or more system outputs 204 (as shown in FIG. 2). In a non-limiting example, the alert may include a notification sent to a caregiver C to inform the caregiver that one or more composite healthcare scores are below a threshold value.

In certain examples, and as described below in further detail with respect to FIGS. 5-9, the one or more recommendations include suggestions generated by the healthcare improvement system 122 to improve the quality, safety, and efficiency of healthcare. In a non-limiting example, the one or more recommendations include suggestions that are sent to a caregiver C to help improve the one or more composite healthcare scores.

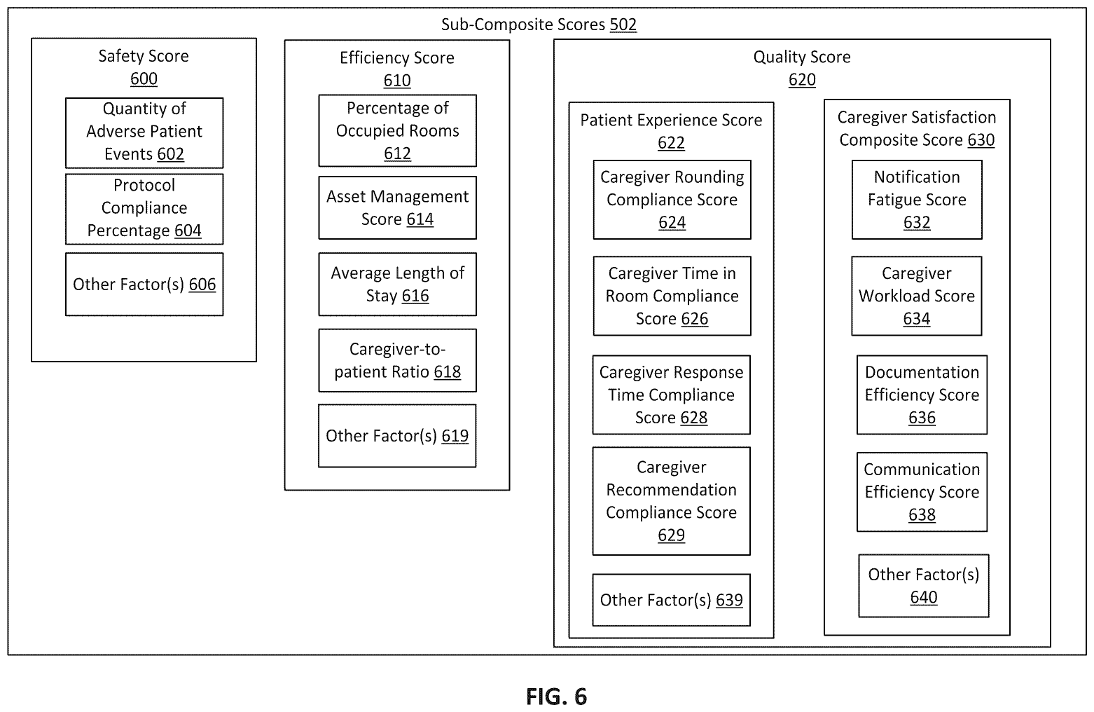

In certain examples, an alert may be sent to a caregiver C with one or more recommendations. In other examples, an alert may be provided to the caregiver C without a recommendation such as when a recommendation is not needed (e.g., when a caregiver C is already completing the task(s) that would be recommended by the healthcare improvement system 122). In other examples, a recommendation may be provided without an alert to improve one or more healthcare composite scores (e.g., the recommendation can be pulled by the caregiver C instead of being pushed to the caregiver C). In further examples, an alert and/or recommendation is not pushed to the caregiver C based on a limited number of alerts that are allowed to be transmitted to the caregiver C during a given time period to reduce alert fatigue (i.e., fatigue that is caused by excessive notifications provided to the caregiver C, which negatively contributes to the notification fatigue score 632 discussed in FIG. 6 below).

In certain examples, the caregiver C may manually request a composite healthcare score that can be provided on demand from the healthcare improvement system 122. The one or more communications devices 102 transmits the request to the healthcare improvement system 122 via the communications network 116. The healthcare improvement system 122 can process the request, generate the alert and/or a recommendation (or update a previous alert and/or recommendation), and transmit the alert and/or recommendation to the communications device 102 via the communications network 116. Furthermore, the caregiver C may provide input to the healthcare improvement system 122 to report progress made when following a recommendation. Further yet, the caregiver C may request an updated composite healthcare score be calculated after following the recommendation.

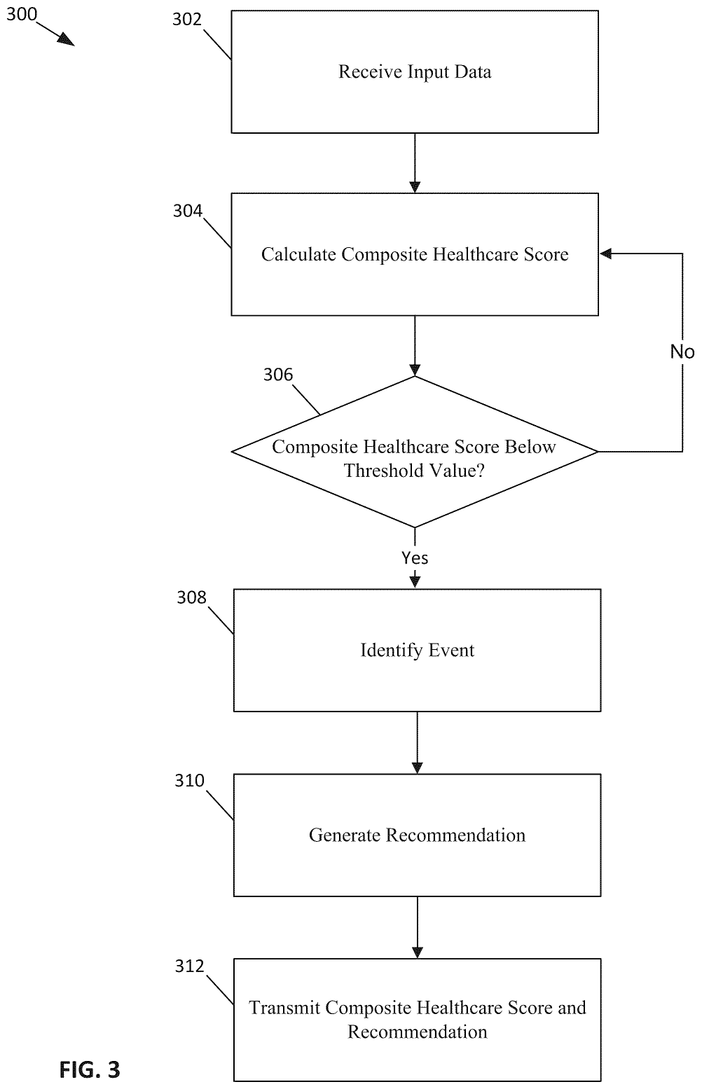

In another example, the healthcare improvement system 122 can proactively generate one or more system outputs 204 and transmit the system outputs 204 to a caregiver C. In certain examples, the healthcare improvement system 122 automatically generates one or more composite healthcare scores, generates an alert and/or recommendation relating to one or more events that reduced the one or more composite healthcare scores below a threshold value, and presents the alert and/or recommendation to a caregiver C (as illustrated and described in further detail with respect to FIG. 3). In certain examples, the healthcare improvement system 122 may automatically update one or more system outputs 204 in real time by continuously monitoring data that is collected within the clinical care environment 10. In certain examples, the healthcare improvement system 122 may automatically provide an update to the composite healthcare scores when one or more actions are completed by one or more caregivers C to follow recommendations that are designed to improve the composite healthcare score.

Advantages of the healthcare improvement system 122 include improving transparency and accountability of clinical care environments 10, allowing patients P to make informed decisions relating to where to solicit healthcare services, improving the quality of healthcare services provided to patients P within the clinical care environment 10 by providing alerts and/or recommendations to caregivers C where improvements can be made, allowing caregivers C observe benchmark measurements compared to other clinical care environments to improve the care that is provided at each clinical care environment 10, improve resource allocation within hospitals to caregivers C or departments that require additional resources, and improving a clinical care environment's compliance with reporting scores to healthcare organizations to meet regulatory requirements and standards set forth by government agencies and accrediting entities. The healthcare improvement system 122 provides improved transparency and accountability to the public by continuously providing composite healthcare scores that measure metrics of the clinical care environment 10 relating to safety, efficiency, and quality of healthcare that removes subjectivity and internal biases.

As further shown in the example provided in FIG. 1, the caregivers C each wear or otherwise carry a tag 104 that is detectable by antennas 114 (also referred to as RTLS readers) positioned throughout the clinical care environment 10. The antennas 114 are fixed reference points that receive wireless signals from the tags 104. The antennas 114 communicate the wireless signals from the tags 104 to the RTLS 110 via the communications network 116. The RTLS 110 uses the data acquired from the antennas 114 to monitor and track the location of the tags 104 (and of the caregivers C) inside the clinical care environment 10. Tracking the location of a caregiver C is useful for determining which actions a caregiver C performs and the time spent to perform each action. The locations of the caregivers C are stored as RTLS data 406 (as shown in FIG. 4) within the RTLS 110. The RTLS data 406 may be transferred to the healthcare improvement system 122 over the communications network 116.

Alternatively, the location of the caregivers C can be monitored by tracking the movement of the communications devices 102. In examples where the communications devices 102 are portable computing devices such as smartphones or table computers carried by the caregiver C, the location of the communications devices 102 can be tracked by various tracking techniques including multilateration of signals between cell towers of a telecommunications network and the communications devices 102, or by using geo-spatial positioning techniques by satellite navigation systems such as the Global Positioning System (GPS).

As shown in FIG. 1, the EMR system 124, also known as electronic health records (EHR) system, is connected to the communications network 116. The EMR system 124 stores the medical history of the patient P. In certain examples, the EMR system 124 generates EMR data 402 (as shown in FIG. 4) that includes information about a patient's P diagnoses (including a current primary diagnosis), past or current medicines that the patient P is taking, physiological variable measurements of the patient P, clinical interventions provided to the patient P, allergies, immunizations, and treatment plans. EMR data 402 pertaining to the patient P is useful for calculating composite healthcare scores because it is indicative of the patient P's experience within the clinical care environment 10, including the pain a patient P experiences, the medical interventions the patient P receives, and the outcome of the medical interventions. The EMR data is illustrated and described in further detail with respect to FIG. 4.

As further shown in the example provided in FIG. 1, a caregiver call system 120 is connected to the communications network 116. The caregiver call system 120 receives requests from the patient P, and can generate and send alerts to a caregiver C to respond to the requests from the patient P. In certain examples, the alert can be routed via the communications network 116 to a communications device 102 used by the caregiver C during their shift. In certain examples, the alert can include a request from the patient P to perform a clinical intervention, such as in response to a triggered alarm. In certain examples, the alert can include information retrieved from the EMR system 124 or from an alarm communication system. In certain examples, the caregiver call system 120 includes one or more caregiver call lights (also known as corridor lights or dome lights) outside of a patient room that indicate when a patient P is requesting help from a caregiver C. The caregiver call system data 408 (shown in FIG. 4) is useful because it provides information relating to a patient P's requests, and period of time that elapses before the request is addressed, and a quantity of requests from the patient P.

As further shown in the example of FIG. 1, an audio system 126 is connected to the communications network 116. The audio system 126 includes a plurality of speakers and microphones positioned throughout the clinical care environment 10 that allow auditory communication between caregivers C, patients P, and the healthcare improvement system 122. In certain examples, the speakers may be included within patient rooms (e.g., on a patient bed 101, along a bed rail 103 of the patient bed 101, or a patient monitoring device such as a spot monitor 105) or within common spaces within the clinical care environment 10 (e.g., hallways, communal spaces, dining areas, and the like). Furthermore, the audio system 126 provides audio information pertaining to system outputs 204 to a caregiver C. In certain examples, the audio system 126 records audio from the caregiver C or patient P that indicates the patient P requires assistance. This may include sounds coming from the patient P that indicate the patient is in distress, verbal communication from the patient P to a caregiver C, or sounds emitted from monitoring equipment, such as a spot monitor 105, that indicate the patient requires assistance.

In certain examples, the clinical care environment systems include one or more monitoring devices that are internet of things (IoT) medical devices. IoT medical devices may be equipped with sensors, connectivity capabilities to a network, and embedded software to collect, transmit, and analyze data related to patient health and medical conditions. Data collected by IoT medical devices can be sent to caregivers C, to other clinical care environment systems, and/or to the healthcare improvement system 122. In certain examples, the patient bed 101 and spot monitor 105 are IoT medical devices that are connected to the communications network 116.

As further shown in the example provided in FIG. 1, a camera system 128 is connected to the communications network 116. The camera system includes a plurality of cameras (e.g., video cameras, surveillance cameras, and the like) positioned throughout the clinical care environment 10 that monitor movements of individuals. The camera system 128 may include cameras positioned within patient rooms or in common spaces. The camera system 128 may be used to record video that is transmitted to the healthcare improvement system 122 to be used to identify actions performed by the caregiver C or movements from patient P.

FIG. 2 schematically illustrates an example of the healthcare improvement system 122. The healthcare improvement system 122 is connected to the communications network 116 via a network interface 206 to facilitate a transfer of data to and from the healthcare improvement system 122. In certain examples, the network interface 206 is a physical network interface that is a hardware component installed within a computer or other device. In certain examples, the network interface 206 is a virtual network interface used in virtualized environments that allows a virtualized system to communicate with a physical network.

The healthcare improvement system 122 receives the input data 400 from one or more of the clinical care environment systems 100 over the communications network 116. Examples the clinical care environment systems 100 are illustrated and described in further detail above with respect to FIG. 1. The clinical care environment systems 100 store the input data and system outputs 204 that are produced by the healthcare improvement system 122 (through a process described below) within a memory.

In certain examples, the healthcare improvement system 122 continuously receives the input data 400 from one or more of the clinical care environment systems 100 and continuously updates the system outputs 204. In certain examples, the healthcare improvement system 122 provides an explanation for why the system outputs 204 was updated. For example, the healthcare improvement system 122 can provide a description of one or more events that were considered in changing the system outputs 204.

A system processor 210 receives and executes instructions from a system memory 202 to perform the functions and aspects described herein. In one example, the system processor 210 receives and executes instructions from a composite healthcare score calculation program 208. In some examples, the instructions received from the composite healthcare score calculation program 208 include processing the input data 400 received over the communications network 116. In some examples, processing the input data 400 includes utilizing one or more artificial intelligence models to generate system outputs 204. For instance, a machine learning algorithm can be trained using input data 400 collected from a large number of patients P. In some examples, the one or more artificial intelligence models use machine learning to improve calculation of one or more composite scores. In further examples, generative artificial intelligence is utilized to generate the system outputs 204 to leverage the composite scores such as to generate recommendations that can be accepted or declined by a user to update one or more settings on one or more medical devices based on one or more composite scores determined by the composite healthcare score calculation program 208. In some further example, the generative artificial intelligence generates answers to user inquiries that leverage the composite scores (e.g., by providing an answer to a questions such as “which unit has the worst patient experience?”). Further examples of applications using the composite healthcare score calculation program 208 are illustrated and described in further detail with respect to FIGS. 7-10.

The system memory 202 includes one or more memories configured to store the composite healthcare score calculation program 208. In certain examples, the system memory 202 stores the input data 400 received via the communications network 116. In such examples, the system memory 202 stores at least one set of input data 400 that includes any combination of the data illustrated and described in further detail with respect to FIG. 4.

In certain examples, the system memory 202 stores the system outputs 204. In such examples, the system memory 202 stores at least some of the system outputs 204 illustrated and described in further detail with respect to FIG. 5.

The system memory 202 can be of various types, including volatile and nonvolatile, removable, and non-removable, and/or persistent media. In some examples, the system memory 202 is an erasable programmable read only memory (EPROM) or flash memory.

The communications device 102 is configured to transmit one or more requests 212 to the healthcare improvement system 122 and receive system outputs 204 via the communications network 116. The one or more requests 212 may be manually generated by a caregiver C when the caregiver C manually requests an alert and/or recommendation from the healthcare improvement system 122 via the one or more communications devices 102 (illustrated and described above with reference to FIG. 1). The communications device 102 stores and displays the system outputs 204 for a caregiver C to review. Examples of communications devices 102 are illustrated and described in further detail with respect to FIGS. 7-9.

FIG. 3 schematically illustrates an example of a method 300 of improving a composite healthcare score. The method 300 can be performed by the healthcare improvement system 122. The method 300 includes a step 302 of receiving data. In some examples, step 302 includes receiving the input data 400 that is generated by the one or more clinical care environment systems 100. The input data 400 is received by the healthcare improvement system 122 from the one or more clinical care environment systems 100 via the communications network 116 as described and illustrated in further detail with above in FIG. 2.

The method 300 includes a step 304 of calculating at least one of the system outputs 204. The composite healthcare scores are calculated by analyzing the input data 400 generated by the one or more clinical care environment systems 100. The input data 400 is analyzed to produce one or more system outputs 204. The system outputs 204 are illustrated and described in further detail with respect to FIG. 5. In certain examples, as shown in FIG. 5, the system outputs 204 include one or more composite healthcare scores 500 that are calculated from one or more sub-composite healthcare scores 502. The system outputs 204, including the composite healthcare scores 500 and the sub-composite healthcare scores 502, are illustrated and described in further detail with respect to FIG. 5.

The method 300 includes a step 306 of comparing the composite healthcare score(s) 500 to a threshold value. If the composite healthcare score(s) 500 are below the threshold value, then the healthcare improvement system 122 proceeds to step 308. If the composite healthcare score(s) 500 are above the threshold value, then the healthcare improvement system 122 continues to monitor the clinical care environment 10 by continuously monitoring and updating the composite healthcare scores as additional input data is received over the communications network 116. In certain alternative examples, the healthcare improvement system 122 may proceed to step 308 even when the composite healthcare score(s) are not below the threshold value to generate one or more system outputs 204 that are designed to improve the healthcare provided to patients P. This may be desirable in instances where few composite healthcare scores are below the threshold value, but the composite healthcare scores could still be improved by implementing one or more system outputs 204.

The method 300 includes a step 308 of identifying at least one event that affected the calculation of the composite healthcare score. In certain examples, the at least one event can include an event that negatively affected the composite healthcare score (e.g., a caregiver C's rounding time between patients was inadequate). In certain examples, the at least one event can include an event that positively affected the composite healthcare score.

The method 300 includes a step 310 of generating a recommendation for improving the composite healthcare score based on the at least one event identified in step 308. In certain examples, step 310 may include generating a recommendation to improve the one or more events that negatively affected the composite healthcare score. In certain examples, step 310 can include encouraging the caregiver to continue performing actions that positively affected the composite healthcare score. An example of a recommendation that can be generated in step 310 is illustrated and described in further detail with respect to FIG. 5.

The method 300 includes a step 312 of transmitting the composite healthcare score and the recommendation to a caregiver C. As illustrated and described above, the composite healthcare score, an alert, and a recommendation can be presented to a caregiver C via a communications device 102 or other devices located within the clinical care environment 10.

FIG. 4 schematically illustrates examples of input data 400 that can be received by the healthcare improvement system 122. As described above, the clinical care environment systems 100 can include the ADT system 108, the RTLS 110, the caregiver call system 120, the EMR system 124, the audio system 126, and the camera system 128. The input data 400 includes EMR data 402 collected by the EMR system 124, ADT system data 404 collected by the ADT system 108, RTLS data 406 collected by the RTLS 110, caregiver call system data 408 collected by the caregiver call system 120, audio system data 410 collected by the audio system 126, and camera system data 412 collected by the audio system 126. The healthcare improvement system 122 analyzes one or more of the EMR data 402, the ADT system data 404, the RTLS data 406, the caregiver call system data 408, the audio system data 410, and the camera system data 412, including any combinations thereof, to generate system outputs 204, which are described and illustrated in further detail with respect to FIGS. 5-8.

The EMR data 402 includes information about a patient P's medical history, including diagnoses, past or current medicines that the patient P is taking, clinical interventions provided to the patient P, recorded vital sign measurements, and other physiological variable measurements, allergies, immunizations, and treatment plans. In certain examples, the caregiver C can enter the EMR data 402 into the EMR system 124 using a communications device 102. In certain examples, the EMR data 402 is utilized to generate system outputs 204 by considering a patient's condition and medical interventions that are provided to the patient.

The ADT system data 404 includes data that tracks patients from their moment of arrival at the clinical care environment 10 until their departure, and can also include relevant patient information such as medical record numbers, names, and contact information. In certain examples, the ADT system data 404 can be utilized to generate system outputs 204 by considering the number of patients within the clinical care environment 10, the time each patient P spent within a clinical care environment 10, the location(s) where each patient P stayed within the clinical care environment 10, and any patient transfers that were made between one or more clinical care environments.

The RTLS data 406 is acquired from the antennas 114 to monitor and track the location of the tags 104 (and the caregivers C) inside the clinical care environment 10. In certain examples, the healthcare improvement system 122 utilizes the RTLS data 406 to generate the system outputs 204 by monitoring the locations of caregivers C, or any other individuals that are equipped with a tag 104, in real time. In certain examples, the locations of individuals that are tracked with the RTLS 110 is monitored in real-time to update the system outputs 204 and provide one or more updated outputs to a caregiver C or patient P. In certain examples, this includes monitoring the location(s) of one or more caregivers C to determine a period of time spent with one or more patients P.

The caregiver call system data 408 includes requests from a patient P and alerts sent to a caregiver C to respond to the request received from the patient P. In certain examples, the healthcare improvement system 122 can analyze the caregiver call system data 408 to analyze communications between a patient P and caregiver C and calculate a period of time between when a patient P requests care and when the caregiver C provides the care that was requested.

The audio system data 410 can be utilized by the healthcare improvement system 122 to analyze sounds emitted by the patient that indicate signs of distress or communications with a caregiver. Furthermore, the audio system data 410 can be utilized by the healthcare improvement system 122 to identify an auditory request from a caregiver C to provide a system output 204. The camera system data 412 can be utilized by the healthcare improvement system 122 to analyze movements of a caregiver C or a patient P.

FIG. 5 schematically illustrates examples of system outputs 204 that are generated by the healthcare improvement system 122. The system outputs 204 include one or more composite healthcare scores 500. In certain examples, the one or more composite healthcare scores 500 are associated with a caregiver C or a patient P, a department within the clinical care environment 10, the clinical care environment 10 as a whole, or a plurality of clinical care environments. In certain examples, the composite healthcare score 500 is a numerical value. The numerical value may include a range of values (e.g., a range from 1-10, 1-100, or any other numerical range).

At least one of the one or more composite healthcare scores 500 can be calculated based on one or more sub-composite healthcare scores 502. The sub-composite healthcare scores 502 include at least one of a safety score 600, an efficiency score 610, and a quality score 620. Input data 400 can be analyzed by the healthcare improvement system 122 to determine each sub-composite healthcare score 502 as illustrated and described below with respect to FIG. 6. Furthermore, the system outputs 204 include alerts 510 and/or recommendations 520 that can be transmitted to the caregiver C. Further yet, the system outputs 204 include an efficiency rating 530 corresponding to an estimated increase in the composite healthcare score 500 per unit time for the caregiver C to implement the at least one recommendation, which is illustrated and described in further detail below.

FIG. 6 illustrates examples of sub-scores and factors that used by the healthcare improvement system 122 to calculate the sub-composite healthcare scores 502. Each of the sub-scores and factors illustrated in FIG. 6 is a key performance indicator (KPI) which is a type of performance measurement. The KPIs illustrated in FIG. 6 evaluate the success of the clinical care environment 10 in providing healthcare to patients.

As shown in FIG. 6, the safety score 600 is calculated based on at least one of a quantity of adverse patient events 602, a protocol compliance percentage 604, and other factors 606. In certain non-limiting examples, the quantity of adverse patient events 602 include a quantity of patient falls, patient mortalities, healthcare-associated infections, preventable incidents, sentinel events, wrong-side surgeries, sepsis events, inappropriate hydration management, medication errors, and other serious safety events. In certain non-limiting examples, the protocol compliance percentage 604 (i.e., a percentage associated with how often a caregiver C, a department, or a clinical care environment 10 properly follows a healthcare protocol) can include a patient fall protocol compliance percentage, a sepsis protocol compliance percentage, a stroke volume index compliance percentage, an empty IV pump compliance percentage, a drug dose error percentage, and a pressure injury prevention protocol compliance percentage. Other factors 606 may include diagnostic errors and equipment failures.

As shown in greater detail in FIG. 6, the efficiency score 610 is calculated based on at least one of a percentage of occupied patient rooms 612, an asset management score 614, an average length of stay 616, a caregiver-to-patient ratio 618, and other factors 619. The percentage of occupied patient rooms 612 is determined by the healthcare improvement system 122 by analyzing the ADT system data 404. The asset management score 614 includes indications where assets were used effectively or ineffectively. In a non-limiting example, examples where assets were used ineffectively may include time spent by a caregiver C finding healthcare devices, such as pumps, vital sign monitors, beds, or communications devices 102. The time spent finding these devices may be calculated by the healthcare improvement system 122 by analyzing the input data 400. Further, the average length of stay 616 and caregiver-to-patient ratio 618 can be calculated by the healthcare improvement system 122 by analyzing the ADT system data 404. The caregiver-to-patient ratio 618 is calculated by comparing the number of available caregivers C to the total patients P admitted to the clinical care environment 10. This ratio may be considered when calculating the caregiver satisfaction score 630 described below. Furthermore, the average length of stay 616 of a patient P may be considered when calculating the quality score 620 described below. Further, the efficiency composite healthcare score may consider other factors 619 relating to the efficiency of caregivers C.

As shown in greater detail in FIG. 6, the quality score 620 includes at least one of a patient experience score 622 which is based on the caregiver's frequency and quality of interactions with the patient. The patient experience score 622 can be calculated based on one or more sub-scores such as a caregiver rounding compliance score 624, a caregiver time in room compliance score 626, a caregiver response time compliance score 628, a caregiver recommendation compliance score 629, and other factors 639.

The sub-scores and the other factors 639 that can be used to calculate the patient experience score 622 can be calculated by the healthcare improvement system 122 based on the input data 400, including the EMR data 402, the RTLS data 406, the caregiver call system data 408, the audio system data 410, the camera system data 412, and the like.

The caregiver rounding compliance score 624 is calculated by the healthcare improvement system 122 by considering the input data 400, including the EMR data 402, the RTLS data 406, the caregiver call system data 408, the audio system data 410, and the camera system data 412. The caregiver rounding compliance score 624 measures a total number of proper patient rotations performed by a caregiver C divided by the total number of patient rotations performed. Furthermore, the healthcare improvement system 122 may also consider other patient experience scores such as a caregiver response time compliance score 628 (i.e., based on a percentage of the number of times a caregiver C responded to a notification from the caregiver call system 120 within a threshold period of time), a time in room compliance score 626 (i.e., based on a percentage of the number of times a caregiver C spent an adequate period of time with a patient P when providing care to a patient P), and other factors 639 relating to a patient's experience within the clinical care environment 10.

As shown in greater detail in FIG. 6, the caregiver satisfaction score 630 includes at least one of a notification fatigue score 632, a caregiver workload score 634, a documentation efficiency score 636, a communication efficiency score 638, and other factors 640. In certain examples, the caregiver satisfaction score 630 is based on a period of time the caregiver spends with each patient P, the number of times a caregiver C visits each patient P, a number of distress events where a patient P requires immediate assistance, the number of times a device alerts the caregiver to provide assistance to a patient (e.g., a patient bed exit, low oxygen saturation, high heart rate, etc.), a number of times the patient P submits a request for assistance via the caregiver call system 120, and a number of messages received via the communications device 102.

The caregiver satisfaction score 630 can include a notification fatigue score 632. The notification fatigue score 632 can be calculated analyzing the input data 400, using the alarm manager 109, to monitor the number of alarms that are transmitted to a caregiver C by one or more devices within the clinical care environment 10. In certain non-limiting examples, these devices include an infusion pump 107, the communications device 102, the caregiver call system 120, a bed exit alarm, the spot monitor 105, the audio system 126, and the camera system 128.

In certain examples, the caregiver satisfaction score 630 includes a caregiver workload score 634. The caregiver workload score is associated with the time the caregiver C spends with each patient P, the number times the caregiver C visits each patient P, the number of alerts transmitted to the caregiver C from the caregiver call system 120, the number of alerts transmitted to the caregiver C from one or more devices within the clinical care environment, and the efficiency of the caregiver C in completing healthcare-related tasks.

The caregiver satisfaction score includes a documentation efficiency score 636 and a communication efficiency score 638. The efficiency of the caregiver C can be measured from certain metrics. In certain examples, these metrics include the caregiver's efficiency in documenting information within the EMR system 124 and the caregiver's efficiency in communicating with the patient P or other caregivers C.

Furthermore, alerts 510 may be transmitted to one or more caregivers C when one or more sub-composite healthcare scores 502 or sub-scores are below a threshold value. For example, the healthcare improvement system 122 may issue an alert when the caregiver recommendation compliance score 629 is less than a threshold value. In another example, the healthcare improvement system 122 may issue an alert when the at least one composite healthcare score is less than a benchmark composite healthcare score (the benchmark composite healthcare score is described in further detail with respect to FIGS. 8-10). In another example, the healthcare improvement system 122 may issue an alert on one or more monitoring devices (e.g., a spot monitor 105) when the at least one monitoring device is associated with a number of events that exceed an event threshold value (i.e., the healthcare improvement system 122 may issue a notification that too many alerts from a monitoring device have been issued within a predetermined period of time, and a recommendation 520 may be implemented as described below to adjust a threshold value that the monitoring device uses for event detection to reduce the quantity of events that are detected by the monitoring device). The alert 510 can include a detailed summary of the composite healthcare score(s) 500 that are below a threshold value and any sub-composite healthcare scores 502 that are below a threshold value.

Referring back to FIG. 5, the system outputs 204 include one or more alerts 510. The alerts 510 are transmitted from the healthcare improvement system 122 to one or more caregivers C via over the communications network 116 to a communications device 102. In certain examples, an alert is transmitted to one or more caregivers C when the composite healthcare score 500 is below the threshold value.

The system outputs 204 include one or more recommendations 520. The recommendations 520 are transmitted from the healthcare improvement system 122 to one or more caregivers C via over the communications network 116 to a communications device 102. In certain examples, one or more recommendations 520 are transmitted to one or more caregivers C when the composite healthcare score 500 is below the threshold value. Furthermore, one or more recommendations 520 may be transmitted to one or more caregivers C when one or more sub-composite healthcare scores 502 are below the threshold value.

The recommendation 520 can include presenting a control on a graphical user interface that allows a caregiver C to view the at least one composite healthcare score and to implement the recommendation 520. In certain examples, this includes adjusting the monitoring device event detection threshold as described above to reduce the quantity of events that are detected by the monitoring device. This improves healthcare by reducing caregiver fatigue caused by excessive alarms, which, in turn, improves the notification fatigue score 632.

The recommendations 520 may include one or more actions that can be performed by the caregiver C to improve the composite healthcare score 500 based on an evaluation of input data 400. In certain examples, the recommendation 520 includes an action that can be performed by the caregiver C to improve a composite healthcare score 500 that is below a threshold value. The recommendation may also include an action that can be performed by the caregiver C to improve a composite healthcare score 500 that is above a threshold value. Furthermore, the recommendation may include a combination of actions that can be performed by a caregiver C to improve composite healthcare scores 500 that are above and below the threshold value.

In certain examples, the healthcare improvement system 122 calculates an efficiency rating 530 corresponding to the recommendation 520. The efficiency rating 530 measures an estimated increase in the composite healthcare score 500 per unit time for the caregiver C to implement the at least one recommendation. Thus, the efficiency rating 530 may be used to order recommendations based on the perceived impact each recommendation 520 has on improving the composite healthcare score 500. For example, a recommendation 520 with the greatest impact on improving the composite healthcare score 500 may be recommended to a caregiver C before another recommendation 520 that has a lesser impact on the composite healthcare score 500.

In certain examples, caregivers C can be monitored after a recommendation is provided to detect whether the caregiver complies with the recommendation 520. Furthermore, a caregiver recommendation compliance percentage 540 can be calculated based on a rate that the caregiver C complies with the recommendation 520. In certain examples, the composite healthcare score 500 and the one or more sub-composite healthcare scores can be updated when a caregiver C complies with at least one recommendation.

FIG. 7 illustrates an example of a communications device 102 displaying a composite healthcare score 500 and a plurality of sub-composite healthcare scores 502 that can be generated by the healthcare improvement system 122. In this example, the composite healthcare score 500 is displayed on a display 118 of the communications device 102. The display 118 can include a touchscreen that displays outputs and receives tactile inputs from the caregiver C. Furthermore, in a non-limiting example, the display 118 shows system outputs 204 relating to a caregiver C that is viewed by selecting the “your rating” icon 708.

The composite healthcare score 500 is displayed over a period of time 704. In a non-limiting example, the period of time 704 shows weekly intervals where the composite healthcare score 500 and sub-composite healthcare scores 502 were calculated. Furthermore, the composite healthcare score may include a rating 706 (e.g., 2 out of 3 stars as shown in FIG. 7) indicating the number of sub-composite healthcare scores 502 that are above the threshold value (e.g., 2 of the 3 sub-composite healthcare scores 502 are above the threshold rating, while the efficiency score 610 falls below the threshold value, which is described in further detail below).

The sub-composite healthcare scores 502 are displayed below the composite healthcare score 500, including the safety score 600, the efficiency score 610, and the quality score 620. In certain examples, the composite healthcare score 500 and the sub-composite healthcare scores 502 are a numerical value within a range of values. In a non-limiting example, the range of values may be from 0-100. In another non-limiting example, the numerical value may be a percentage, where zero percent indicates a complete lack of compliance with clinical care standards and one hundred percent indicates perfect compliance with clinical care standards.

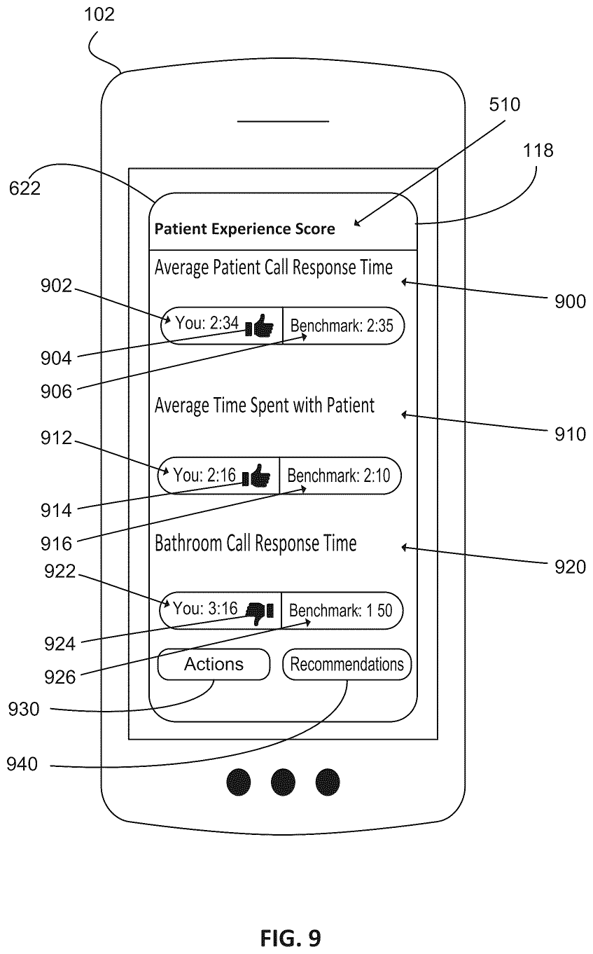

Indicators are displayed to indicate whether the sub-composite healthcare scores 502 are positive, neutral, or negative scores. For example, a positive indicator 716 is included to indicate the quality score 620 is a positive score (e.g., 70%). A neutral indicator 714 is included to indicate the safety score 600 is a neutral score (e.g., 62%). A negative indicator 712 is included to indicate the efficiency score 610 is a negative score (e.g., 46%). In certain examples, the sub-composite healthcare scores 502 are determined to be positive, neutral, or negative by comparing the sub-composite scores to one or more benchmark measurements (which are illustrated and described in further detail with respect to FIGS. 8-9). In certain examples, the negative indicator 712 indicates one or more scores are below a threshold value. Furthermore, the indicators may include a visual indication to visually indicate whether the sub-composite healthcare score 502 is positive, neutral, or negative. For example, a color indicator such as green text or green highlighting for a positive score (e.g., positive indicator 716), black text or no highlighting for a neutral score (e.g., neutral indicator 714), and red text or red highlighting for a negative score (e.g., negative indicator 712). Also, a recommendations icon 702 is displayed that allows the user to view the recommendations 520 relating to the composite healthcare score 500 and the sub-composite healthcare scores 502.

FIG. 8 illustrates an example of a communications device 102 displaying the recommendations 520 for improving the composite healthcare scores illustrated in FIG. 7. In this example, the recommendations 520 are displayed on a display 118 of the communications device 102 when the recommendations icon 702 of FIG. 7 is selected. The display 118 can include a touchscreen that displays outputs and receives tactile inputs from the caregiver C.

The recommendations 520 can include, for example, a recommendation to integrate patient safety parameters and/or to integrate operating room parameters. The recommendations 520 each include an option 802 to automatically implement the recommendation. As an example, selecting option 802 can include automatically adjusting a threshold value that a monitoring device uses to detect events to reduce a quantity of events detected by the monitoring device, and thereby reduce alarm fatigue when the monitoring device issues a number of alarms that exceed a threshold. The threshold value that the monitoring device uses to detect events may be adjusted by the alarm manager 109.

Furthermore, the recommendation 520 or alert 510 may include a benchmark comparison 804 to other benchmark clinical care environments. A benchmark clinical care environment is a different clinical care environment that includes similar characteristics to the clinical care environment of the communications device 102. In certain examples, the healthcare improvement system 122 identifies at least one benchmark clinical care environment based on one or more of the similar characteristics. For example, the characteristics that the healthcare improvement system 122 uses to identify the at least one benchmark clinical care environment can include at least one of a number of rooms available to patients, a square-foot area of the at least one benchmark clinical care environment, an age of the at least one benchmark clinical care environment, a location of the at least one benchmark clinical care environment, patient demographics within the at least one benchmark clinical care environment, or specialty care offered within the at least one benchmark clinical care environment.

The healthcare improvement system 122 can generate a benchmark composite healthcare score corresponding to the at least one benchmark clinical care environment. Further yet, the healthcare improvement system 122 issues an alert when the at least one composite healthcare score (or sub-composite healthcare score) is less than the benchmark composite healthcare score. The benchmark comparison 804 shown in FIG. 8 is an example of an alert that may be issued when the at least one composite healthcare score is less than the benchmark composite healthcare score (e.g., the efficiency score 610 was 20% lower than similar benchmark clinical care environments).

FIG. 9 illustrates an example of a communications device 102 displaying comparisons between a caregiver C's actions and actions of a benchmark caregiver in a similar clinical care environment. In this example, the benchmark measurements 900, 910, 920 are displayed on a display 118 of the communications device 102. The display 118 can include a touchscreen that displays outputs and receives tactile inputs from the caregiver C.

The display 118 shows example metrics relating to the patient experience score 622 of FIG. 6. The alert includes three example benchmark measurements 900, 910, 920.

A first example benchmark measurement 900 relates to the average patient call response time (i.e., the average time that elapses between when a patient P requests care using the caregiver call system 120 and when the caregiver C responds to the request). In the first example benchmark measurement 900, a first caregiver score 902 is listed that states the average patient call response time for the caregiver C is two minutes and thirty-four seconds. A first caregiver indicator 904 is displayed to indicate that the first caregiver score 902 is better than the first benchmark caregiver score 906 relating to the average patient call response time for the benchmark caregiver (e.g., two minutes and thirty-five seconds).

A second example benchmark measurement 910 relates to the average time a caregiver spends with a patient when providing care to the patient. In the second example benchmark measurement 910, a second caregiver score 912 is listed that states the average time the caregiver C spends with the patient P is two minutes and sixteen seconds. A second caregiver indicator 914 is displayed that indicates the second caregiver score 912 is better than the second benchmark caregiver score 916 relating to the average time spent with a patient for the benchmark caregiver (e.g., two minutes and ten seconds).

A third example benchmark measurement 920 relates to an average time for a caregiver C to respond to a patient P when the patient transmits a request for aid in using the bathroom. In the third example benchmark measurement 920, a third caregiver score 922 provides the average required for the caregiver to respond to a bathroom call is three minutes and fifteen seconds. A third caregiver indicator 924 is displayed to indicate the third caregiver score 922 is worse than the third benchmark caregiver score 926 relating to an average time required for the benchmark caregiver to respond to the bathroom call (e.g., one minute and fifty seconds).

Furthermore, the alert 510 can include an actions icon 930 and a recommendations icon 940 for requesting additional information from the healthcare improvement system 122 or requesting a recommendation 520. In certain examples, the actions icon 930 allows the user to request an update to the system output 204 or implement one or more recommendations 520 to improve the one or more composite healthcare scores relative to the one or more benchmark measurements 900, 910, 920. Through clinical studies based on data gathered from device inputs across a plurality of clinical care environments such as hospitals, correlations with a statistical level of confidence in predicting an outcome based on a threshold of each sub-composite healthcare score 502 (e.g., see FIG. 6) are used to recommend what steps to take to improve patient outcomes. For example, when the data shows that a 5 minute or less response time to a request from a patient results in a 5 of 5 star rating when patients fill out an experience survey, the recommendation can include to respond more quickly to a request from a patient when a caregiver's average response time is over 5 minutes. The recommendations icon 940 allows a caregiver C to view one or more recommendations 520 for improving the one or more composite healthcare scores relative to the one or more benchmark measurements 900, 910, 920.

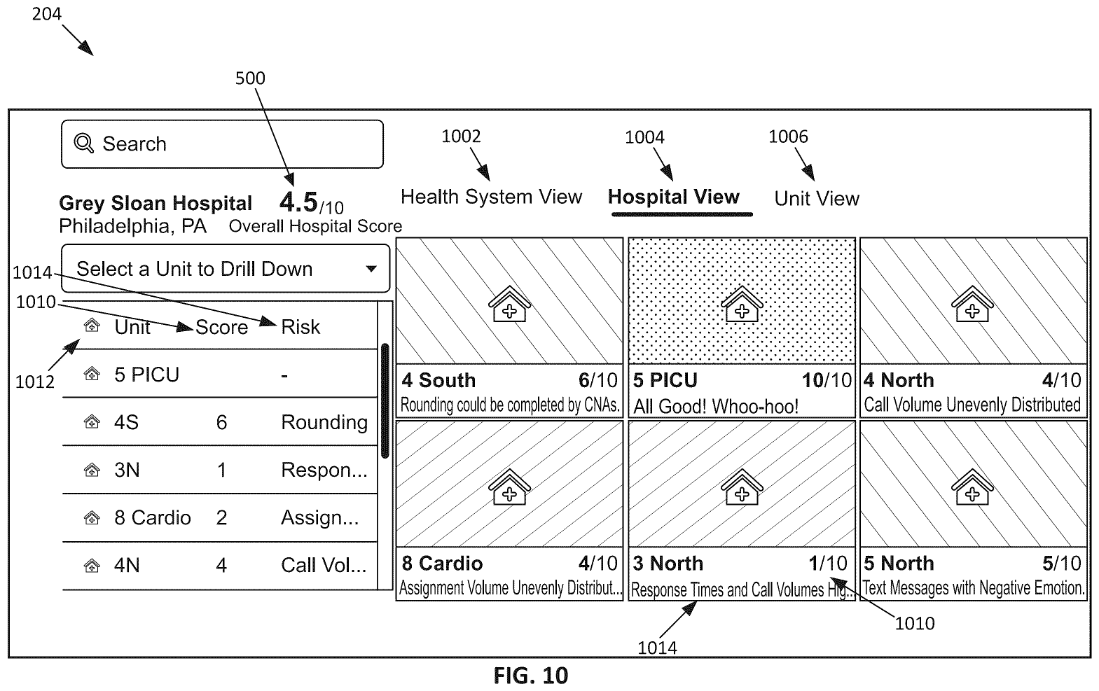

FIG. 10 illustrates an example of composite healthcare scores being calculated for different hierarchical levels of a clinical care environment and compared to hierarchical levels of a benchmark clinical care environment. An example of a system output 204 is shown that includes composite healthcare scores relating to a health system (i.e., a plurality of clinical care environments 10), a hospital view 1004 (i.e., a single clinical care environment) and a unit view 1006 (i.e., a unit or department within a clinical care environment 10). Additionally, a composite healthcare score can be calculated for individual caregivers C in the health system.

The example illustrated in FIG. 10 shows a plurality of departments 1012 within a clinical care environment 10. The example includes a composite healthcare score 500 for a clinical care environment 10 (e.g., a score of 4.5/10). The healthcare improvement system 122 calculates a department composite healthcare score 1010 for each department 1012 (e.g., a score of 6/10 for department 4 South, and a score of 10/10 for department 5 PICU). Furthermore, the healthcare improvement system 122 displays one or more risks 1014 (i.e., one or more issues within each department that could be improved) for each department composite healthcare score 1010. In certain examples, the risks 1014 are included for departments 1012 having a department composite healthcare score 1010 that is below a threshold value or below a benchmark value pertaining to a benchmark department score in a benchmark clinical care environment.

In certain examples, a visual indicator may be included to visually indicate whether the composite healthcare score 500 or department composite healthcare score 1010 is perceived as a positive, neutral, or negative score relative to another benchmark clinical care environment. For example, color highlighting may be used as the visual indicator. The color highlighting may include green highlighting for scores that are perceived as being positive relative to another benchmark facility, yellow highlighting for scores that are perceived as being neutral relative to another benchmark facility, and red highlighting for scores that are perceived as being negative relative to another benchmark facility.

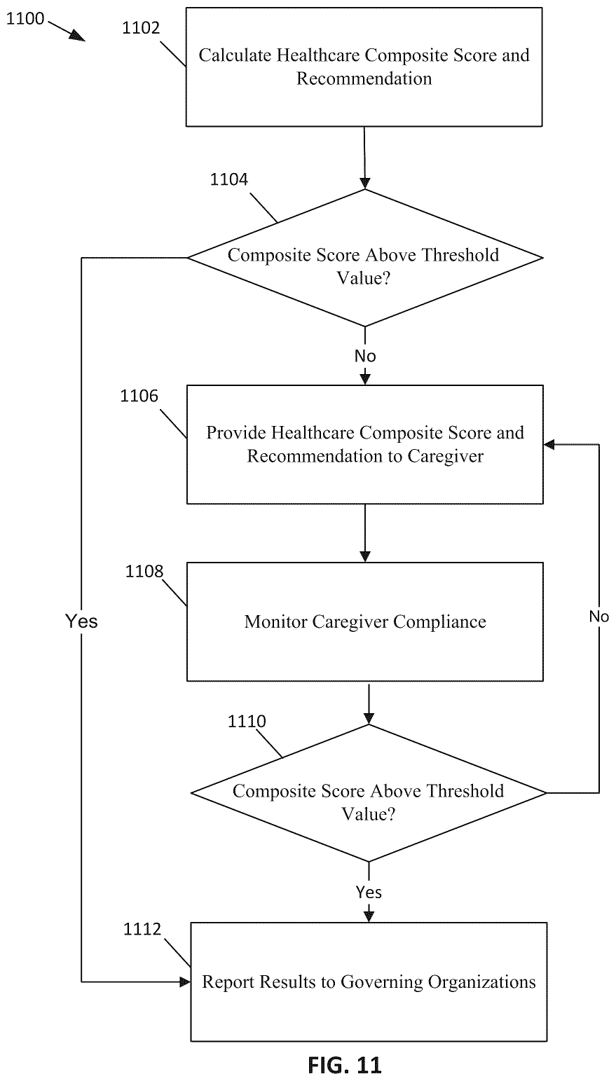

FIG. 11 illustrates an example of a method 1100 of reporting the one or more composite scores. In some examples, the method 1100 can be performed by the healthcare improvement system 122. The method 1100 includes a step 1102 of calculating a composite healthcare score 500. Methods for calculating the composite healthcare score 500, and various sub-composite healthcare scores that are considered when calculating the composite healthcare score, are illustrated and described in further detail with respect to FIGS. 1-10.

The method 1100 includes a step 1112 of evaluating whether the composite healthcare score is above a threshold value. If the composite healthcare score is above the threshold value, then the healthcare improvement system 122 may proceed to a step 1110 of reporting the composite healthcare score to a governing organization. If the composite healthcare score is below the threshold value, then the healthcare improvement system 122 may proceed by enabling the caregiver C to submit changes to improve the composite healthcare score 500, which is described in further detail below with respect to steps 1104, 1106, and 1108.

The method 1100 includes a step 1104 of providing the composite healthcare score 500 and the recommendation 520 to a caregiver C. In certain examples, the composite healthcare score 500 and the recommendation 520 are transmitted to the communications device 102 operated by the caregiver C from the healthcare improvement system 122 over the communications network 116.

The method 1100 includes a step 1106 of monitoring caregiver compliance with the recommendation provided in step 1104. As described above with reference to FIGS. 5-6, the healthcare improvement system 122 may calculate a caregiver recommendation compliance percentage 540 and issue an alert when the caregiver recommendation compliance percentage 540 is less than a threshold value. Furthermore, the healthcare improvement system 122 may issue an alert when the at least one composite healthcare score is less than a benchmark composite healthcare score (the benchmark composite healthcare score is described in further detail with respect to FIGS. 8-10). Further yet the healthcare improvement system 122 may issue a monitoring device alert on one or more monitoring devices (e.g., a spot monitor 105) when the at least one monitoring device is associated with a number of events that exceed an event threshold value (i.e., the healthcare improvement system 122 may issue an alert notifying a caregiver C that the caregiver C is receiving too many alerts from a monitoring device, and the monitoring device event detection threshold can be adjusted to reduce the quantity of events that are detected by the monitoring device).

The method 1100 includes a step 1108 of determining whether the composite healthcare score is above a threshold value. If the composite healthcare score is above the threshold value, then the composite healthcare score is reported in step 1110 to a governing organization such as the Centers for Medicare and Medicaid Services, the Joint Commission, the National Committee for Quality Assurance, and Press Ganey. In certain examples, the composite score may be provided to the caregiver again to repeat the steps 1104 and 1106 when the composite score remains below the threshold value. Thus, the healthcare improvement system 122 may initially calculate a composite healthcare score that is below the threshold value, provide recommendations to a caregiver C to improve the composite healthcare score, determine whether the composite healthcare score has increased above the threshold value, and repeat the steps 1104 and 1106 when the composite healthcare score remains below the threshold value.

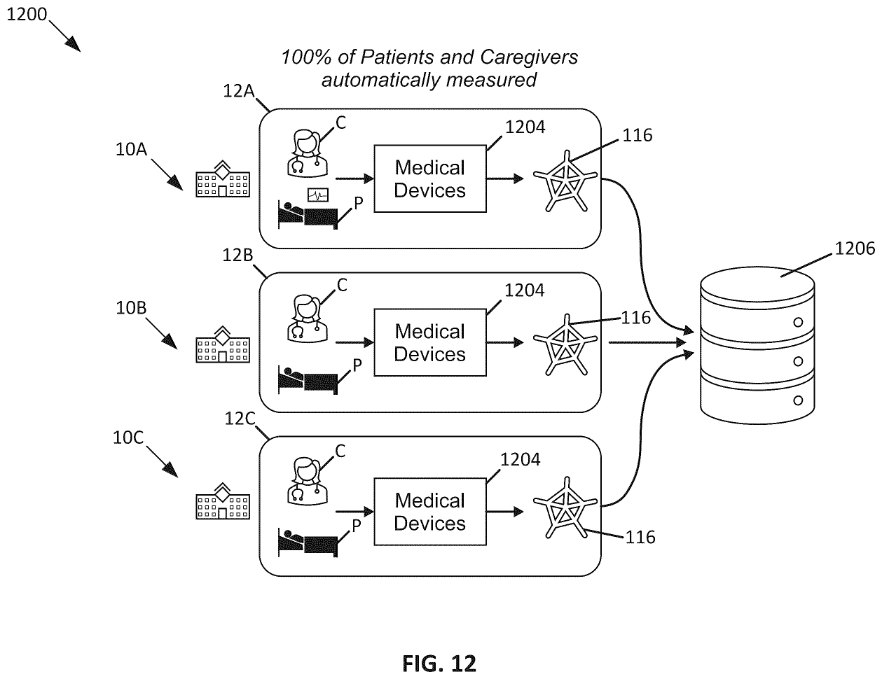

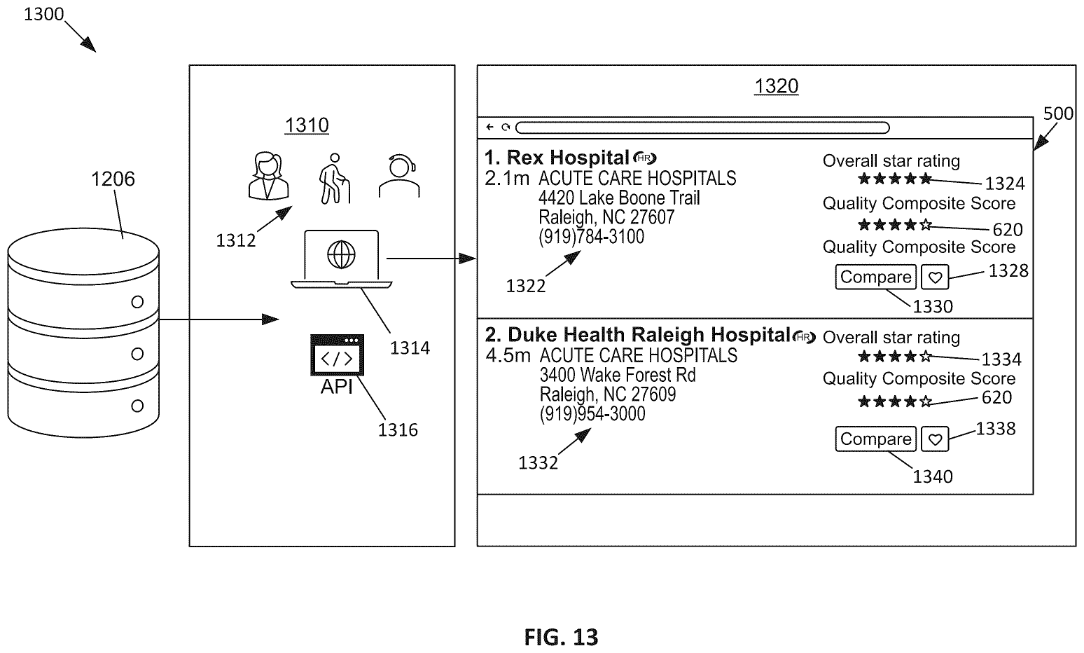

FIG. 12 illustrates a schematic view of clinical care environments 10A, 10B, 10C collecting data using medical devices 1204 to store in a database 1206. In certain examples, the clinical care environments 10A, 10B, 10C are part of a system of clinical care environments 1200 that each collect the data from one or more medical devices 1204. The clinical care environments 10A, 10B, 10C each include at least one patient environment 12A, 12B, 12C. The medical devices 1204 collect the data from one or more patients P or caregivers C and transfer the data to a database 1206 via a communications network 116. In certain examples, the medical devices include monitoring devices that are illustrated and described above with reference to FIG. 1. In certain examples, the medical devices include IoT medical devices as described above with respect to FIG. 1 that can transmit data to the database 1206 via a communications network 116. The data stored in the database 1206 is made available for the general public to access, which is described in further detail below with respect to FIG. 13.

FIG. 13 illustrates a schematic view of a process 1300 for accessing the data collected and stored in FIG. 12 in a web portal 1320 to enable the view of one or more composite healthcare scores 500 associated with one or more clinical care environments. One or more users 1310 can access the data using one or more devices 1314 that are connected to the database via the internet. The one or more devices 1314 may use an application programming interface 1316 (API) to request and exchange information between the database, the user, and the web portal 1320 that displays information that is accessible to the public.

The web portal 1320 includes one or more composite healthcare scores 500 corresponding to a clinical care environment 1322, 1332. Each of the clinical care environments 1322, 1332 includes an overall rating 1324, 1334, a quality score 620, a compare icon 1330, 1340, and a favorite icon 1328, 1338. In certain examples, the overall rating 1324, 1334 and the quality score 620 include a star rating on a scale of 1-5 with one star corresponding to a rating or score and five stars corresponding to an excellent rating or score.

The compare icon 1330, 1340 includes a functionality to compare one clinical care environment to another clinical care environment using information stored within the database 1206. Furthermore, the favorite icon 1328, 1338 allows a user 1310 to save a specific clinical care environment as a “favorite” option for healthcare services, and information pertaining to a favorited clinical care environment can be accessed at a later time within a list of example clinical care environments where the favorite icon was selected.

The web portal 1320 provides benchmarking that is accessible to the public, and that provides useful information to potential patients that is collected from medical device data rather than subjective measurements such as patient surveys. Furthermore, government organizations can rely on scores generated from medical device data to appropriately reimburse clinical care environments for healthcare services provided through programs such as Medicare.

The systems and methods described herein provide significant technical advantages. For example, the healthcare improvement system 122 improves visibility of healthcare services and the quality of the services within clinical care environments 10 by automatically generating composite healthcare scores relating to input data 400 received from one or more clinical care environment systems 100. Furthermore, the healthcare improvement system 122 is a practical application in healthcare technology that conveys specific alerts and/or recommendations, which may include graphical and/or visual information, in a specific way at a communications device 102 to assist caregivers C and provide greater visibility of healthcare services and quality to patients P. The healthcare improvement system 122 may automatically implement one or more recommendations to improve the functioning of healthcare systems, such as to adjust an event detection threshold value for one or more monitoring devices to reduce the number of alarms produced by the monitoring devices, reduce caregiver fatigue, improve caregiver satisfaction, and improve a caregiver's ability to provide quality care to patients P.

The description and illustration of one or more embodiments provided in this application are not intended to limit or restrict the scope of the invention as claimed in any way. The embodiments, examples, and details provided in this application are considered sufficient to convey possession and enable others to make and use the best mode of claimed invention. The claimed invention should not be construed as being limited to any embodiment, example, or detail provided in this application. Regardless of whether shown and described in combination or separately, the various features (both structural and methodological) are intended to be selectively included or omitted to produce an embodiment with a particular set of features.

Having been provided with the description and illustrations of the present application, one skilled in the art may envision variations, modifications, and alternate embodiments falling within the spirit of the broader aspects of the claimed inventions and the general inventive concepts embodied in this application that do not depart from the broader scope.

The various embodiments described above are provided by way of illustration only and should not be construed to be limiting in any way. Various modifications can be made to the embodiments described above without departing from the true spirit and scope of the disclosure.

What is claimed is:

1. A system for improving healthcare, the system comprising:
at least one monitoring device connected to a network;
at least one processing device; and
a memory device storing instructions which, when executed by the at least one processing device, cause the at least one processing device to:
receive data from the at least one monitoring device, wherein the data includes a plurality of actions performed by one or more caregivers;
calculate at least one composite healthcare score by analyzing the plurality of actions performed by the one or more caregivers;
identify at least one of the plurality of actions that negatively impacted the at least one composite healthcare score;
generate at least one recommendation for improving the at least one composite healthcare score based on the at least one of the plurality of actions that negatively impacted the at least one composite healthcare score; and
present a control on a graphical user interface that allows a caregiver of the one or more caregivers to view the at least one composite healthcare score and implement the at least one recommendation.

2. The system of claim 1, wherein the at least one monitoring device includes at least one of a real-time location system, a caregiver call system, a camera system, an audio system, a patient bed, a spot monitor, or an infusion pump.

3. The system of claim 1, wherein the at least one of the plurality of actions that negatively impacted the at least one composite healthcare score is identified when the at least one composite healthcare score is less than a threshold value.

4. The system of claim 1, wherein the at least one composite healthcare score is calculated based on one or more sub-composite healthcare scores.

5. The system of claim 4, wherein the one or more sub-composite healthcare scores include at least one of a safety score, an efficiency score, a quality score, and a caregiver satisfaction score.

6. The system of claim 5, wherein the safety score is calculated based on at least one of a quantity of adverse patient events and a protocol compliance percentage.

7. The system of claim 5, wherein the efficiency score is calculated based on at least one of a percentage of occupied patient rooms, an asset management score, an average length of admission, and a caregiver-to-patient ratio.

8. The system of claim 5, wherein the quality score includes at least one of a patient experience score based on the caregiver's frequency and quality of interactions with the patient, a caregiver rounding compliance percentage, a caregiver time in room compliance percentage, and a caregiver response time compliance percentage.

9. The system of claim 5, wherein the at least one caregiver satisfaction score includes at least one of a notification fatigue score, a caregiver workload score, a documentation efficiency score, or a communication efficiency score.

10. The system of claim 1, wherein the instructions, when executed by the at least one processing device, further cause the at least one processing device to:
calculate a caregiver recommendation compliance percentage based on a rate that the caregiver complies with the at least one recommendation.

11. The system of claim 10, wherein the instructions, when executed by the at least one processing device, further cause the at least one processing device to:
issue an alert when the caregiver recommendation compliance percentage is less than a threshold value.

12. The system of claim 1, wherein the at least one composite healthcare score is generated for at least one of a clinical care environment, at least one department within the clinical care environment, and at least one caregiver assigned to the at least one department.

13. The system of claim 12, wherein the instructions, when executed by the at least one processing device, further cause the at least one processing device to:
identify at least one benchmark clinical care environment including one or more benchmark clinical care environment characteristics that are within a range of benchmark threshold values;
generate a benchmark composite healthcare score corresponding to the at least one benchmark clinical care environment; and
issue the alert when the at least one composite healthcare score is less than the benchmark composite healthcare score.

14. The system of claim 13, wherein the one or more benchmark clinical care environment characteristics include at least one of a number of rooms available to patients, a square-foot area of the at least one benchmark clinical care environment, an age of the at least one benchmark clinical care environment, a location of the at least one benchmark clinical care environment, patient demographics within the at least one benchmark clinical care environment, or specialty care offered within the at least one benchmark clinical care environment.

15. The system of claim 1, wherein the instructions, when executed by the at least one processing device, further cause the at least one processing device to:
calculate an efficiency rating corresponding to the at least one recommendation, wherein the efficiency rating measures an estimated improvement in the at least one composite healthcare score per unit of time for the caregiver to implement the at least one recommendation.

16. A method for improving healthcare, the method comprising:
receiving data from at least one monitoring device, wherein the data includes a plurality of actions performed by one or more caregivers;
calculating at least one composite healthcare score by analyzing the plurality of actions performed by the one or more caregivers;
identifying at least one of the plurality of actions that negatively impacted the at least one composite healthcare score;
generating at least one recommendation for improving the at least one composite healthcare score based on the at least one of the plurality of actions that negatively impacted the at least one composite healthcare score; and
presenting a control on a graphical user interface that allows a caregiver of the one or more caregivers to view the at least one composite healthcare score and to implement the at least one recommendation.

17. The method of claim 16, further comprising:
monitoring the caregiver to detect when the caregiver complies with the at least one recommendation; and
calculating a caregiver recommendation compliance percentage based on a rate that the caregiver complies with the at least one recommendation.

18. The method of claim 16, further comprising:
identifying at least one benchmark clinical care environment including one or more benchmark clinical care environment characteristics that are within a range of benchmark threshold values;
generating a benchmark composite healthcare score corresponding to the at least one benchmark clinical care environment; and
issuing an alert when the at least one composite healthcare score is less than the benchmark composite healthcare score.

19. The method of claim 16, further comprising:
identifying the at least one monitoring device associated with the plurality of actions;
issuing a monitoring device alert when the at least one monitoring device is associated with a number of events that exceed an event threshold value; and
adjusting an event detection threshold value for the at least one monitoring device to reduce a quantity of events that are detected by the at least one monitoring device.

20. A non-transitory computer readable storage medium storing instructions, which when executed by a computing device, causes the computing device to:
receive an alert containing a notification that at least one composite healthcare score is below a threshold value;
receive at least one recommendation for improving the at least one composite healthcare score based on an evaluation of monitoring device data;
provide the at least one recommendation to a caregiver;
update the at least one composite healthcare score when the caregiver complies with the at least one recommendation; and
provide a second alert to the caregiver when the at least one composite healthcare score increases above the threshold value.
